(19) United States
(12) Patent Application Publication
(10) Pub. No.: US 2026/0204349 A1
(43) Pub. Date: Jul. 16, 2026

(54) GENE EXPRESSION PREDICTION FROM WHOLE SLIDE IMAGES

(71) Applicant: Verily Life Sciences LLC, Dallas, TX (US)

(72) Inventors: Ronnachai Jaroensri, South San Francisco, CA (US); Po-Hsuan Cameron Chen, Palo Alto, CA (US); David F. Steiner, South San Francisco, CA (US); Yun Liu, South San Francisco, CA (US); Ellery Wulczyn, South San Francisco, CA (US)

(73) Assignee: Verily Life Sciences LLC, Dallas, TX (US)

(21) Appl. No.: 19/135,587

(22) PCT Filed: Dec. 20, 2023
(86) PCT No.: PCT/US2023/085122

§ 371 Date: Jun. 4, 2025

Related U.S. Application Data
(60) Provisional application No. 63/476,751, filed on Dec. 22, 2022.

(51) Int. Cl. G16B 25/10; G06V 10/26; G06V 10/40; G06V 10/82; G06V 20/69; G16B 40/20

(52) U.S. Cl. CPC G16B 25/10; G06V 10/273; G06V 10/40; G06V 10/82; G06V 20/695; G16B 40/20; G06V 2201/03

(57) ABSTRACT

One example method for gene expression prediction from whole slide images includes receiving one or more images of stained tissue; generating a set of image segments from the one or more images, each image segment comprising a group of pixels from an image of the one or more images; determining, for each image segment and using a first trained machine learning (“ML”) model, a vector of feature values; determining, using a second trained ML model and based on each of the vectors of feature values, a predicted gene expression; and outputting the predicted gene expression.

### CROSS-REFERENCES TO RELATED APPLICATIONS

The present application claims priority to U.S. Provisional Application No. 63/476,751, filed on Dec. 22, 2022, the disclosure of which is herein incorporated by reference in its entirety for all purposes.

### FIELD

The present application generally relates to detecting gene expression in pathology images and more particularly relates to gene expression prediction from whole slide images.

### BACKGROUND OF THE INVENTION

Interpretation of tissue samples to determine the presence of cancer requires substantial training and experience with identifying features that may indicate cancer. Typically, a pathologist will receive a slide containing a slice of tissue and examine the tissue to identify features on the slide and determine whether those features likely indicate the presence of cancer, e.g., a tumor. In addition, the pathologist may also identify features, e.g., biomarkers, that may be used to diagnose a cancerous tumor, that may predict a risk for one or more types of cancer, or that may indicate a type of treatment that may be effective on a tumor.

### BRIEF SUMMARY OF THE INVENTION

Various examples are described for gene expression prediction from whole slide images. One example method includes receiving one or more images of stained tissue; generate a set of image segments from the one or more images, each image segment comprising a group of pixels from an image of the one or more images; determining, for each image segment and using a first trained machine learning (“ML”) model, a vector of feature values; determining, using a second trained ML model and based on each of the vectors of feature values, a predicted gene expression; and outputting the predicted gene expression.

One example system for gene expression prediction from whole slide images includes a non-transitory computer-readable medium; one or more processors in communication with the non-transitory computer-readable medium, the one or more processors configured to execute processor-executable instructions stored in the non-transitory computer-readable medium configured to cause the one or more processors to receive one or more images of stained tissue; generate a set of image segments from the one or more images, each image segment comprising a group of pixels from an image of the one or more images; determine, for each image segment and using a first trained machine learning (“ML”) model, a vector of feature values; determine, using a second trained ML model and based on each of the vectors of feature values, a predicted gene expression; and output the predicted gene expression.

One example non-transitory computer-readable medium comprising processor-executable instructions configured to cause one or more processors to receive one or more images of stained tissue; generate a set of image segments from the one or more images, each image segment comprising a group of pixels from an image of the one or more images; determine, for each image segment and using a first trained machine learning (“ML”) model, a vector of feature values; determine, using a second trained ML model and based on each of the vectors of feature values, a predicted gene expression; and output the predicted gene expression.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 shows an example system for gene expression prediction from whole slide images;

FIG. 2 shows an example of an image of a slide that includes a stained tissue sample suitable for use with systems and methods for gene expression prediction from whole slide images according to this disclosure;

FIGS. 3-4 show example system for gene expression prediction from whole slide images;

FIG. 5 shows an example method for gene expression prediction from whole slide images;

FIG. 6 shows an example computing device suitable for use with various systems and methods for gene expression prediction from whole slide images.

### DETAILED DESCRIPTION OF THE INVENTION

Examples are described herein in the context of gene expression prediction from whole slide images. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application-and business-related constraints, and that these specific goals will vary from one implementation to another and from one developer to another.

The treatment of cancer is complex, with protocols that differ based on the many subtypes. These subtypes are traditionally determined by examining the tumor under a microscope. However, molecular testing, such as gene expression profiling (GEP) has also become an important source of information to inform treatment in some cases. For example, OncotypeDx, a GEP test, can identify patients with low-risk cancer to help them make the decision to forgo chemotherapy and thus avoid the associated toxicities and costs of such treatment. But despite its benefit, patients face challenges to access this type of molecular testing. For example, the tests cost thousands of dollars, require coordination of tissue shipping, and can take several weeks to return results. Furthermore, genetic testing can require a substantial amount of tissue, which may be an issue of particular importance for small tumors or low tumor content specimens. Thus, detecting such information from pathology images may be of significant value for patients.

However, detecting gene expression markers, such as the gene encoding estrogen receptor (“ER”), in pathology samples can be difficult and subject to interpretation by the pathologist reviewing the sample. For example, the detection process can involve immunohistochemistry (“IHC”) staining the sample, which the pathologist then views in a magnified image of the sample, whether under a microscope directly or via a captured image of the sample. From the IHC-stained sample, the pathologist can identify features in the sample that indicate the presence (or absence) of particular biomarkers. IHC staining, however, can be substantially more expensive than other types of stains, e.g., hematoxylin and eosin (“H&E”) staining, which may be more readily available. But in addition to staining difficulties, stained images of pathology samples can be very large, often containing 108 pixels or more. Thus, manually reviewing such images for signs of cancer can be time-consuming and requires significant training and expertise.

To help facilitate identification of potential tumors within a pathology sample, this disclosure provides example systems and methods to predict gene expression from whole slide high-resolution color images (“whole slide images”). Further, because a pathology sample may be sectioned into multiple slides, examples may operate on multiple images captured from the same pathology sample.

An example system for predicting gene expression from whole slide images, e.g., in stained tissue samples taken from a human breast, involves using trained machine learning (“ML”) models to analyze one or more digitized images of the stained sample. To digitize the sample, a thin slice of tissue may be stained and positioned on a slide, where it is imaged, typically using optical magnification. This process may then be repeated for additional slices of tissue. The captured image is then analyzed to identify foreground pixels from background pixels, with foreground pixels representing the stained tissue. The foreground pixels are then segmented into a number of image segments and a subset of these image segments are sampled randomly. The selected image segments are then inputted into one trained ML model, which performs a feature analysis on the image segments. The features identified by this ML model are not human interpretable; however, they provide a vector of values that can be inputted into a second stage ML model.

After generating the vectors for each of the selected image segments across the captured image(s), the vectors for the image(s) are then fed into a second trained ML model. The second ML model includes an attention-based deep multiple-instance learning model. This second ML model accepts the large number of feature values generated by the first ML model and generates a gene expression prediction that includes a single prediction value, which can be employed by a pathologist to determine a clinical ER status and potential patient outcomes.

By employing the two-stage ML architecture, including the attention-based deep multiple-instance learning model, the example system is able to quickly and accurately determine a level of gene expression within the pathology sample, even across multiple images of different portions of the sample. This can help reduce or eliminate the reliance on expensive, invasive, and slow genetic testing that may otherwise be employed. This may help provide a faster diagnosis to the patient, reducing patient anxiety, and a lower cost for the patient and health care provider.

This illustrative example is given to introduce the reader to the general subject matter discussed herein and the disclosure is not limited to this example. The following sections describe various additional non-limiting examples and examples of gene expression prediction from whole slide images.

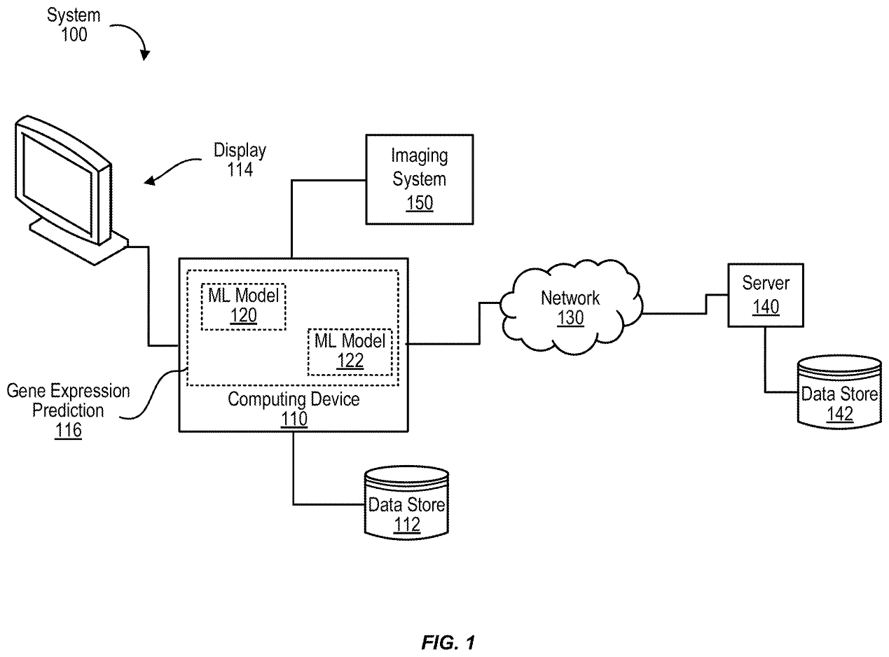

Referring now to FIG. 1, FIG. 1 shows an example system 100 for gene expression prediction from whole slide images. The system 100 includes an imaging system 150 that is connected to a computing device 110. The computing device 110 has gene expression prediction software 116, which includes two ML models 120-122 stored in memory, and is connected to a display 114, a local data store 112, and to a remote server 140 via one or more communication networks 130. The remote server 140 is, in turn, connected to its own data store 142.

The imaging system 150 includes a microscope and camera to capture images of pathology samples. Imaging system 150 in this example is a conventional pathology imaging system that can capture digital images of tissue samples, stained or unstained, using broad-spectrum visible light. The imaging system 150 can include (for example) a microscope (e.g., a light microscope) and/or a camera. In some instances, the camera is integrated within the microscope and the microscope can include a stage on which the portion of the sample (e.g., a slice mounted onto a slide) is placed, one or more lenses (e.g., one or more objective lenses and/or an eyepiece lens), one or more focuses, and/or a light source. The camera may be positioned such that a lens of the camera is adjacent to the eyepiece lens. In some instances, a lens of the camera is included within image collection system 104 in lieu of an eyepiece lens of a microscope. The camera can include one or more lenses, one or more focuses, one or more shutters, and/or a light source (e.g., a flash). In this example, the imaging system 150 captures images at 10× magnification, corresponding to about 1 micron (10⁻⁶ m) per pixel, though any suitable magnification may be employed. The computing system 110 receives digital images from the imaging system 150 corresponding to a particular tissue sample and provides them to the ML models 120-122 to predict gene expression within the tissue sample.

The tissue samples can include, but are not limited to, a sample collected via a biopsy (such as a core-needle biopsy), fine needle aspirate, surgical resection, or the like. In one scenario, a tissue sample will be prepared for imaging within the conventional imaging system 150, such as by obtaining one or more thin slices of tissue taken from a patient, staining the slices with a suitable stain (e.g., H&E), and positioning them on corresponding slides, which are then inserted in sequence into the imaging system 150. The imaging system 150 then captures images of the stained samples (referred to as “stained images”) and provides them to the computing device 110. A set of images may be then generated by the image imaging system and each image of the set of images may correspond to different portions of the biological sample.

After receiving the captured stained image or multiple captured stained images, the computing device 110 may store the image(s) in the data store 112. It then executes the gene expression prediction software 116 on the images for a particular biological sample. It should be appreciated that, while images from multiple different biological samples may be available in the data store 112, only the images corresponding to a particular biological sample are processed together.

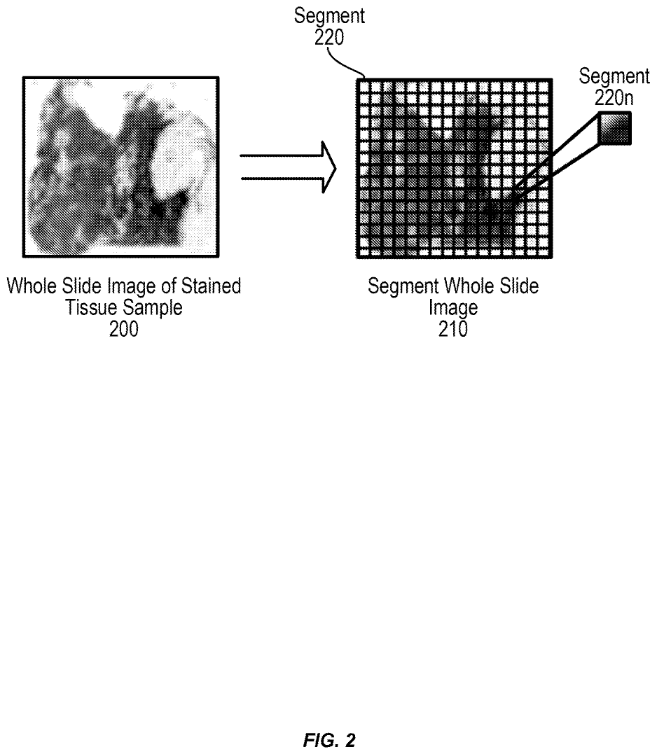

Initially, for each image corresponding to the biological sample, the gene expression prediction software 116 identifies foreground and background portions of each image and ignores the background portions. It then segments the foreground portions of the image(s) into segments of 224×224 pixels, though any suitably sized segments may be used. This segmenting process is depicted in FIG. 2. FIG. 2 shows an example whole slide image of a stained tissue sample. As discussed above, the tissue is stained with an H&E stain, though any suitable stain may be employed. The slide is then segmented into a segmented whole slide image 210 with multiple segments 220. As discussed above, the system may first remove background pixels before segmenting, though in this example, the entire whole slide image 200 is segmented and any segments having a threshold number of background pixels are ignored.

Some or all of the remaining segments are then provided to the first ML model 120, which generates a set of feature values for each segment. The feature values for all of the segments analyzed by the first ML model 120 are then combined into a single two-dimensional matrix and inputted into the second ML model 122, which generates and outputs a single gene expression prediction value.

While in this example, the entire process occurs on the local computing device 110 and imaging system 150, such an arrangement is not needed. For example, an example system may omit the imaging system 150. Instead, the computing device 110 could obtain whole slide images from its data store 112 or from the remote server 140. Alternatively, while gene expression prediction software 116 is executed at the computing device 110, in some examples, the whole slide images may be provided to the remote server 140, which may execute gene expression prediction software 116, including suitable ML models, e.g., ML models 120-122. Thus, the system shown in FIG. 1 may, according to different examples, provide gene prediction analysis in setting having suitable imaging devices or by receiving images of pathology tissue from a third party for processing, including in a cloud environment provided by a remote server 140.

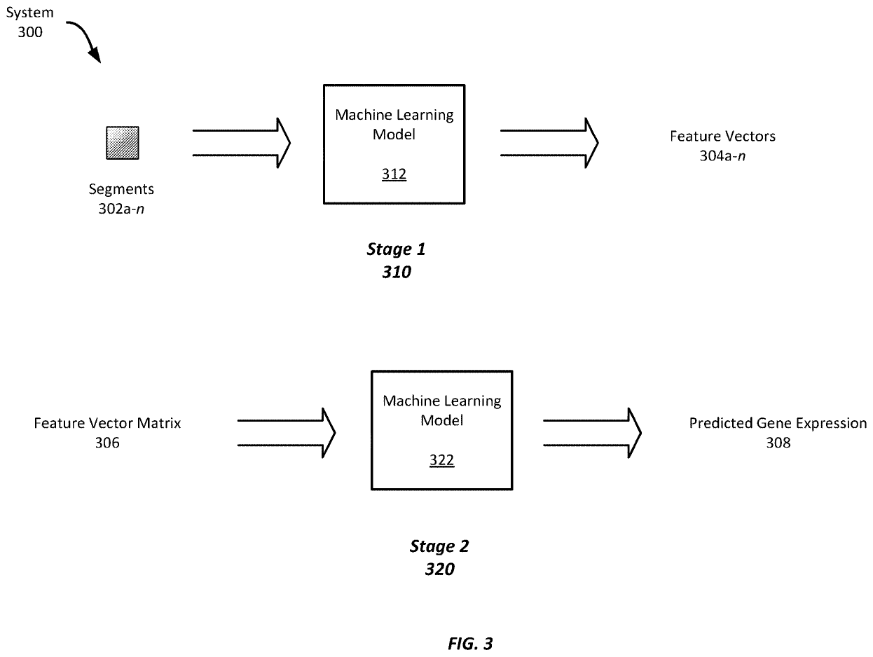

Referring now to FIG. 3, FIG. 3 shows an example system for gene expression prediction from whole slide images. In this example, the system includes two trained ML models 312, 322 arranged in sequence. As discussed above with respect to FIG. 1, one or more images corresponding to a tissue sample are segmented and only segments that include sufficient foreground pixels are retained. In this example, the segments from all images for a particular tissue sample are then aggregated and a subset of 16,384 segments is randomly selected for analysis. It should be appreciated that any suitable number of segments may be selected, up to and including all segments from the image(s), in different examples. Further, while this example aggregates all segments from all images for a tissue sample, some examples, may sample segments from each image. In addition, other sampling techniques may be employed. Finally, some examples may employ all segments from all images.

After selecting a set of segments 302a-n, gene expression prediction software, such as shown in FIG. 1, executes the first ML model 312 and provides the selected segments 302a-n. In this example, the first ML model 312 is an artificial neural network (“NN”), such as a residual NN. However, any other suitable artificial NN may be used, including convolutional NNs, long short-term memory recurrent NNs, etc. The first ML model 312 has been trained to determine feature values from image segments using self-supervised training on a suitable set of training pathology images.

From each inputted segment 302a-n, the first ML model 312 generates a vector 304a-n of 2,048 feature values providing a high-level feature representation of the segment. While this example outputs vectors 304a-n having 2,048 feature values per segment, other examples may generate vectors of different size. In general, these feature values are not meaningful to humans, but provide a high-level feature description of the respective segment that are meaningful to the first and second ML models 120-122 based on their respective training processes. Thus, after processing the segments 304a-n, the gene expression prediction software accumulates 16,384 vectors 304a-n, which provides a two-dimensional vector (or matrix) having 16,384 rows and each row having 2,048 features values. The two-dimensional feature vector matrix 306 is then provided to the second ML model 322.

The second ML model 322 in this example is an attention-based deep multiple instance learning model that includes multiple fully connected layers and an attention mechanism. For each row in the feature vector matrix 306, the second ML model 322 employs an attention mechanism to dynamically apply weights to the different rows of the matrix 306 based on the corresponding feature values. The final layer of the second ML model 122 outputs a value representing the gene expression prediction 308. In this example, the gene expression prediction 308 is not scaled and thus may have arbitrary size; however, some examples may normalize the gene expression prediction to a desired range, such as a real number from zero to one.

The gene expression prediction 308 is then provided as an output on the computing device 110. However, in some examples, the gene expression prediction 308 may be stored in the data store 112 and associated with the one or more whole slide images. As discussed above, some examples may perform gene expression prediction at a remote server 140, such as provided by a cloud service provider, a health care provider, or a third-party test provider. The determined gene expression prediction 308 may then be stored in the data store 142 at the remote server 140 and associated with the one or more whole slide images. In some examples, the gene expression prediction 308 may be stored in a data store and associated with a patient or patient's profile.

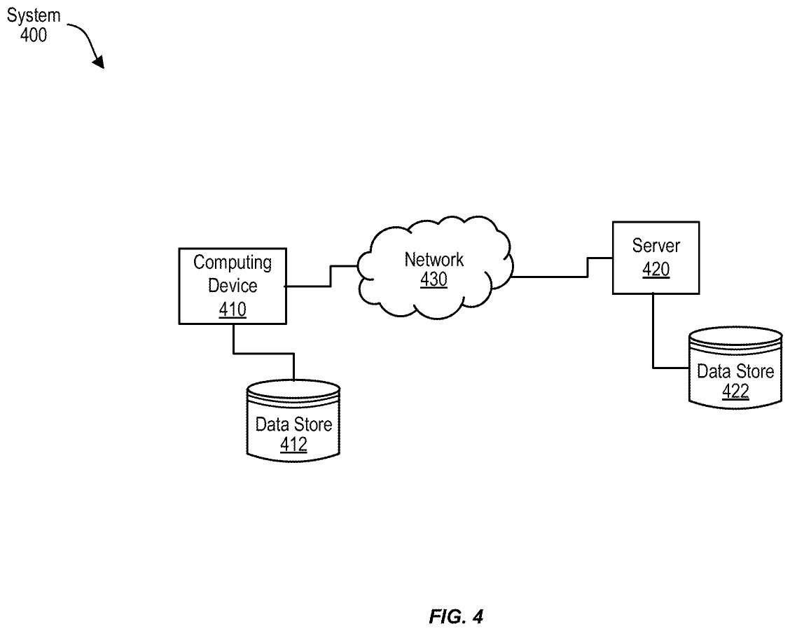

Referring now to FIG. 4, FIG. 4 shows an example system 400 for gene expression prediction from whole slide images. The example system 400 includes a computing device 410 that has access to a data store 412 and is connected to server 420 and its data store 422 via network 430. In this example, the computing device 410 accesses digitized pathology samples from data store 412 and provides them to the server 420 for analysis, such as using the system 300 described above with respect to FIG. 3. After completing the analysis, the server 420 returns the results to the computing device 410 or stores them in data store 422 for later retrieval, e.g., by medical personnel.

In this example, the server 420 is maintained by a medical provider, e.g., a hospital or laboratory, while the computing device 410 is resident at a medical office, e.g., in a pathologist's office. Thus, such a system 400 may enable medical providers at remote locations to obtain and stain tissue samples and provide those samples to a remote server 420 that can provide the analysis of the samples. However, it should be appreciated that example systems according to this disclosure may only include computing device 410, which may perform the analysis itself without communicating with a remote computing device.

To implement systems according to this example system 400, any suitable computing device may be employed for computing device 410 or server 420. Further, while the computing device 410 in this example accesses digitized pathology samples from the data store 412, in some examples, the computing device 410 may be in communication with an imaging device that captures images of pathology samples. Such a configuration may enable the computing device to capture an image of a pathology sample and immediately process it using suitable ML models, or provide it to a remote computing device, e.g., server 420, for analysis.

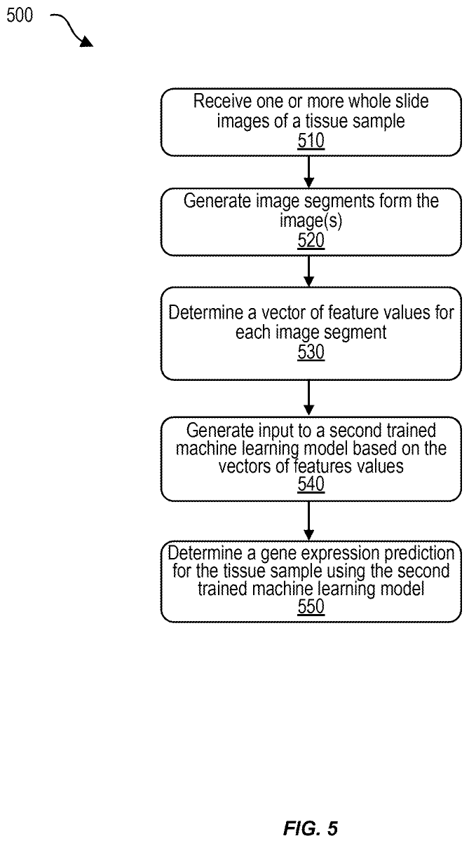

Referring now to FIG. 5, FIG. 5 shows an example method 500 for gene expression prediction from whole slide images. The method 500 will be described with respect to the example system 100 shown in FIG. 1; however, any suitable system according to this disclosure may be used, such as the system 400 shown in FIG. 4.

At block 510, the computing device 110 receives one or more images of stained tissue. In this example, the computing device 110 receives one or more images slides having portions of an H&E-stained tissue sample, though in some examples, any suitable stain may be employed. As discussed above, a tissue sample may be taken from a patient and one or more slices from the sample may be prepared and imaged using an imaging system, such as imaging system 150. Thus, a single tissue sample may result in multiple different images, though some examples may include only a single slide from a tissue sample. The slices of tissue may be each be stained and imaged to provide corresponding whole slide images that are received by the computing device 110.

In this example, the images are received from the imaging system 150; however, in some examples, the images may be received from a local data store 112 or from a remote computing system, such as a remote server 140. Alternatively, the one or more whole slide images may be provided to a remote computing device, such as remote server 140, which receives the images and may store them in a data store 142.

At block 520, the computing device 110 generates a set of image segments from the one or more images. As discussed above with respect to FIG. 2, each image may be segmented into multiple segments of any suitable size, such as segments of 240×240 pixels. Further, background pixels within the images may be identified and either excluded from the segmenting process or segments with more than a threshold number of background pixels may be excluded from the set of image segments. Thus, in this example, the set of image segments includes segments with all foreground pixels or with at least a threshold number of foreground pixels.

In some examples, the set of image segments may be reduced in size by selecting only a subset of image segments having a suitable number of foreground pixels. In this example, the computing device 110 randomly selects 16,384 image segments to serve as the set of image segments. The remaining image segments may then be discarded.

At block 530, a first trained ML model 120 determines a vector of feature values for each image segment in the set of image segments. In this example, each of the 16,384 image segments are provided, in sequence, to the first trained ML model 120. The first trained ML model 120 then generates a corresponding vector of feature values for each image segment. In this example, the first trained ML model 120 generates vectors having 2,048 feature values, though any suitable number of feature values may be generated according to various example. Further, it should be appreciated that the feature values do not represent human-interpretable values. As discussed above, the feature values may represent any suitable information within the image segments as determined by the first ML model that may be provided to a second trained ML model for subsequent gene expression prediction.

At block 540, the computing device 110 generates input to a second trained ML model 122 based on the vectors of feature values generated by the first trained ML model 120 at block 530. In this example, the computing device 110 generates a two-dimensional matrix of values with each row of the matrix including one vector of the vectors generated at block 530. Thus, the input matrix includes all of the vectors determined at block 530.

At block 550, the second trained ML model 122 determines a gene expression prediction for the tissue sample based on the input matrix generated at block 540. As discussed above with respect to FIG. 3, a second trained ML model 322 may use an attention-based deep multiple instance learning model to determine the gene expression prediction 308. In this example, the gene expression prediction 308 is an unbounded value of any size; however, some examples may provide a gene expression prediction 308 within a defined range, such as a real number from zero to one.

After determining the gene expression prediction 308, the computing device 110 outputs the gene expression prediction 308, such as by displaying it on the display 114 or storing it within a data store 112. In some examples, the gene expression prediction 308 may be transmitted to another computing device, such as to a remote server 140 or to any other computing device. For example, if gene expression prediction is performed at a server 420, the gene expression prediction 308 may be provided to a remote computing device 410.

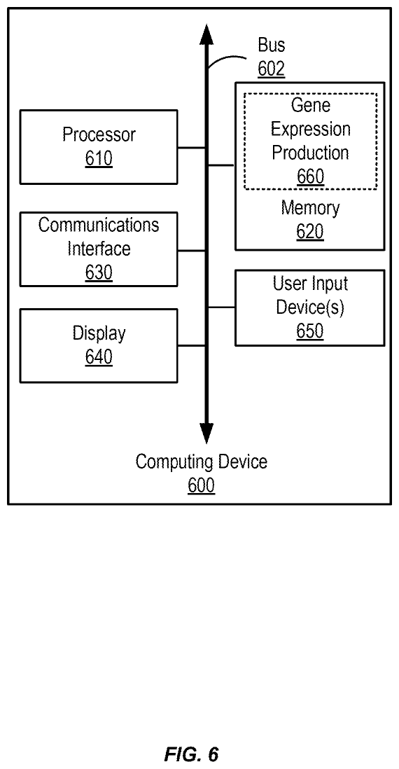

Referring now to FIG. 6, FIG. 6 shows an example computing device 600 suitable for use in example systems or methods for gene expression prediction from whole slide images according to this disclosure. The example computing device 600 includes a processor 610 which is in communication with the memory 620 and other components of the computing device 600 using one or more communications buses 602. The processor 610 is configured to execute processor-executable instructions stored in the memory 620 to perform one or more methods for gene expression prediction from whole slide images according to different examples, such as part or all of the example method 500 described above with respect to FIG. 5. In this example, the memory 620 includes a gene expression prediction system 660, such as the example system shown in FIG. 1 or FIG. 3. In addition, the computing device 600 also includes one or more user input devices 650, such as a keyboard, mouse, touchscreen, microphone, etc., to accept user input; however, in some examples, the computing device 600 may lack such user input devices, such as remote servers or cloud servers. The computing device 600 also includes a display 640 to provide visual output to a user.

The computing device 600 also includes a communications interface 640. In some examples, the communications interface 630 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, the methods and systems may also be implemented as specifically configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random-access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, that may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

What is claimed is:

1. A method comprising:
receiving one or more images of stained tissue;
generating a set of image segments from the one or more images, each image segment comprising a group of pixels from an image of the one or more images;
determining, for each image segment and using a first trained machine learning (“ML”) model, a vector of feature values;
determining, using a second trained ML model and based on each of the vectors of feature values, a predicted gene expression; and
outputting the predicted gene expression.

2. The method of claim 1, wherein the set of images segments is a second set of image segments, and wherein generating the second set of images segments comprises:
segmenting the image into a first set of image segments, each image segment comprising pixels corresponding to stained tissue; and
selecting a subset of image segments for the second set of image segments.

3. The method of claim 2, wherein selecting the subset of image segments comprises randomly selecting the subset of image segments.

4. The method of claim 1, wherein the first trained ML model comprises a convolutional neural network.

5. The method of claim 1, wherein the first trained ML model comprises a residual neural network.

6. The method of claim 1, wherein the second trained ML model comprises an attention-based deep multiple-instance learning model.

7. The method of claim 1, wherein the one or more images comprises a plurality of images, and wherein generating the set of image segments comprises generating image segments for each image of the plurality of images.

8. The method of claim 7, wherein generating the set of image segments further comprises randomly selecting a subset of the image segments for each image of the plurality of images.

9. The method of claim 7, wherein generating the set of image segments further comprises excluding one or more image segments comprising a threshold number of background pixels.

10. A system comprising:
a non-transitory computer-readable medium;
one or more processors in communication with the non-transitory computer-readable medium, the one or more processors configured to execute processor-executable instructions stored in the non-transitory computer-readable medium configured to cause the one or more processors to:
receive one or more images of stained tissue;
generate a set of image segments from the one or more images, each image segment comprising a group of pixels from an image of the one or more images;
determine, for each image segment and using a first trained machine learning (“ML”) model, a vector of feature values;
determine, using a second trained ML model and based on each of the vectors of feature values, a predicted gene expression; and
output the predicted gene expression.

11. The system of claim 10, wherein the one or more images comprises a plurality of images, and wherein generating the set of image segments comprises generating image segments for each image of the plurality of images.

12. The system of claim 11, wherein generating the set of image segments further comprises randomly selecting a subset of the image segments for each image of the plurality of images.

13. The system of claim 11, wherein generating the set of image segments further comprises excluding one or more image segments comprising a threshold number of background pixels.

14. The system of claim 10, wherein the first trained ML model comprises a residual neural network.

15. The system of claim 10, wherein the second trained ML model comprises an attention-based deep multiple-instance learning model.

16. A non-transitory computer-readable medium comprising processor-executable instructions configured to cause one or more processors to:
receive one or more images of stained tissue;
generate a set of image segments from the one or more images, each image segment comprising a group of pixels from an image of the one or more images;
determine, for each image segment and using a first trained machine learning (“ML”) model, a vector of feature values;
determine, using a second trained ML model and based on each of the vectors of feature values, a predicted gene expression; and
output the predicted gene expression.

17. The non-transitory computer-readable medium of claim 16, wherein the one or more images comprises a plurality of images, and wherein generating the set of image segments comprises generating image segments for each image of the plurality of images.

18. The non-transitory computer-readable medium of claim 17, wherein generating the set of image segments further comprises randomly selecting a subset of the image segments for each image of the plurality of images.

19. The non-transitory computer-readable medium of claim 17, wherein generating the set of image segments further comprises excluding one or more image segments comprising a threshold number of background pixels.

20. The non-transitory computer-readable medium of claim 16, wherein the second trained ML model comprises an attention-based deep multiple-instance learning model.
